(19) United States
(12) Patent Application Publication
(10) Pub. No.: US 2002/0156439 A1
(43) Pub. Date: Oct. 24, 2002

(54) COLLECTION CONTAINER ASSEMBLY

(76) Inventor: MICHAEL J. ISKRA, BRIDGEWATER, NJ (US)

Correspondence Address: RICHARD J RODRICK, BECTON DICKINSON AND COMPANY, 1 BECTON DRIVE, FRANKLIN LAKES, NJ, 074171880

(21) Appl. No.: 08/928,272

(22) Filed: Sep. 12, 1997

(51) Int. Cl. A61M 1/00; B65D 81/00; B04B 3/00; C02F 1/38; B01D 33/15; B01D 33/27

(52) U.S. Cl. 604/317; 600/573; 210/782

(57) ABSTRACT

The present invention is a collection container assembly comprising a container with a false bottom having a rounded open end and wherein the external dimensions of the container are substantially the same as a standard-sized blood collection tube but with a reduced internal volume.

### BACKGROUND OF THE INVENTION

1. Field of the Invention

This invention relates to a specimen collection container assembly and more particularly to a collection container for collecting biological fluid specimens where a small quantity of fluid may be collected and retained in the container while maintaining a container size sufficient to be easily accommodated and/or compatible with standard clinical equipment and instrumentation.

2. Description of Related Art

Blood samples and other biological fluid specimens are routinely taken and analyzed in hospital and clinical situations for various medical purposes. Collection, handling and testing of these samples typically requires the use of various medical testing instruments. As the blood and fluid specimens are usually collected in a standard sized collection tube, the medical instruments used to test the samples are designed to accommodate these standard sized collection tubes.

Conventional blood collection tubes used in most clinical situations are elongated cylindrical containers having one end closed by a semi-spherical or rounded portion and an opposed open end. The open end may be sealed by a resilient cap or stopper. The tube defines a collection interior which collects and holds the blood sample. The most common size of these blood collection tubes are designed to accommodate approximately 10 ml of blood or other biological fluid samples. Illustrative of such blood collection tubes is the VACUTAINER® brand s blood collection tube sold by Becton, Dickinson and Company, 1 Becton Drive, Franklin Lakes, N.J. (registered trademark of Becton, Dickinson and Company).

A phlebotomist or other medical technician typically obtains a specimen of the patient's blood in the tube by techniques well known in the art. The tube is then appropriately labeled and transferred from the site of collection to a laboratory or other location where the contents of the tube are analyzed. During collection and analysis the tube may be supported by various medical instruments. The plasma or serum derived therefrom is processed and analyzed either manually, semi-automatically or automatically. In some cases, the specimen must first be dispensed from the collection tube to a sample test tube or cuvette.

In certain situations it is only necessary to obtain a small quantity of blood or other biological fluid specimens. These situations may include pediatric, or geriatric patients and other instances where large blood samples are not required. Small quantities of blood cannot be easily collected in standard collection tubes as described above because the sample level in such containers would not be adequate for retrieval prior to analysis. Such small quantities of fluids also have a tendency to significantly evaporate when stored in larger containers, thus concentrating the chemical and enzymatic constituents therein. This may result in erroneous analytical results and could possibly affect the diagnosis and treatment given to the patient. Therefore, it is desirable to employ small-volume containers which substantially inhibit evaporation for the storage and delivery of minute fluid samples in the laboratory.

Although various fluid containers are available for this purpose, their small overall size and shape make it difficult for the phlebotomist or other medical technicians to handle and manipulate the tubes. Furthermore, such small dimension tubes are generally incompatible with most handling and testing instrumentation. For example, their use in conventional storage racks or those designed for loading into automatic chemical analyzers is substantially precluded because of their small dimensions. Certain automated chemical analyzers are capable of utilizing standardized conventional specimen containers as a means for introducing a patient's specimen into the analyzer. However, they are generally not equipped to handle specimen containers designed to hold small quantities of fluid.

In addition, as the labels placed on most blood collection tubes are read by optical instrumentation such as bar code readers, conventional bar code labels may be too large to be supported on the small volume tubes.

Various specimen containers such as those incorporating a “false bottom” have been proposed to achieve decreased volume capacity in conjunction with standard external dimensions. However, these various specimen containers are not compatible with standard clinical equipment and instrumentation due to their design. In particular, these specimen containers have false bottoms with a generally flat, planar bottom end and a circular shaped opening.

In clinical use, it is desirable for such specimen collection containers to have rounded bottom configurations that closely simulate a standard-sized blood collection tube configuration instead of planar bottoms. Rounded bottom configurations facilitate compatibility with clinical equipment and instrumentation.

Therefore there is a need to provide a specimen collection container assembly for collecting blood samples and other biological fluid specimens of relatively small volumes where the assembly may be accommodated and/or compatible with standard clinical equipment and/or instrumentation and where the integrity of the sample and specimens are maintained during storage and transport.

### SUMMARY OF THE INVENTION

The present invention is a collection assembly comprising a container. The container preferably comprises an open top portion, a lower bottom portion and a sidewall extending from the open top portion to the lower bottom portion. Lower bottom portion comprises a closed bottom end or true bottom and an annular skirt extending from the closed bottom end to a rounded stop end. The annular skirt provides a false bottom effect to the assembly and the rounded stop end provides a means for allowing the container to be compatible with standard clinical equipment and instrumentation.

The true end may be the same or different material than the container and may be integral with the container or may be a discrete member. Additionally, the true end may be arcuate in shape to provide an internal volume for specimen collection having at least a partially rounded true bottom portion, or may be conical in shape.

Alternatively, the annular skirt may extend from the closed bottom end to a fully rounded second closed bottom end and may further comprise a cap or a stopper.

Preferably, the external dimensions of the container are about the same as a standard-sized or full draw blood collection container assembly. A standard-sized blood collection container assembly has an outer diameter of about 13 to about 16 millimeters, a length of about 75 to about 100 millimeters and an internal volume of about 6 to about 10 milliliters.

Most preferably, the assembly of the present invention can be either evacuated or non-evacuated. Desirably, the assembly is made from thermoplastic polymers. Most desirably, the assembly is made from polyethylene terephthalate, polypropylene, polyethylene, polyethylene napthalate or copolymers thereof.

An advantage of the assembly of the present invention is that it provides a full-draw blood collection container assembly having a reduced internal volume but with external dimensions about the same as a standard-sized blood collection container assembly.

A further advantage of the assembly of the present invention is that it provides a specimen collection container which is universally compatible with various clinical equipment and instrumentation. In particular, the assembly of the present invention does not require any external adapters and the like to be attached to the assembly to be compatible with various clinical equipment and instrumentation.

The assembly of the present invention may be easily handled by equipment configured to handle standard-sized blood collection tubes having standard external dimensions.

Most notably, is that the assembly of the present invention provides a blood collection container having full draw external dimensions but with a reduced internal volume as compared to standard-sized full draw blood collection tubes.

The assembly of the present invention therefore addresses the need for a full-draw size low-volume blood collection container assembly that presents the external dimensions of a standard-sized blood collection tube.

The assembly of the present invention may be used to reliably collect small samples of blood or biological fluids and to maintain the integrity of the samples is during storage and transport as compared to using standard-sized blood collection tubes. In addition, the assembly of the present invention can also be accommodated by standard-sized blood collection, transportation, storage, and diagnostic equipment.

Most notably, is that the assembly of the present invention provides a rounded bottom configuration that closely simulates a standard-sized blood collection tube with a fully rounded bottom. This particular feature in conjunction with all of the features of the container, distinguishes it from the specimen containers that have flat planar bottoms.

The assembly of the present invention is also compatible with existing instrumentation, labels, and bar code readers and obviates the need for new instrumentation and handling devices or procedures that would be required for smaller or varying sized tubes or tubes with flat planar bottoms.

### DESCRIPTION OF THE DRAWINGS

FIG. 1 is a perspective view of a false bottom specimen tube of the prior art.

FIG. 2 is a longitudinal sectional view of the tube of FIG. 1 taken along line 2-2 thereof.

FIG. 3 is a perspective view of the assembly of the present invention.

FIG. 4 is a longitudinal sectional view of the assembly of FIG. 3 taken along line 4-4 thereof.

FIG. 5 is a perspective view of an alternate embodiment of the present invention.

FIG. 6 is perspective view of an alternate embodiment of the present invention.

### DETAILED DESCRIPTION

The present invention may be embodied in other specific forms and is not limited to any specific embodiment described in detail which is merely exemplary. Various other modifications will be apparent to and readily made by those skilled in the art without departing from the scope and spirit of the invention. The scope of the invention will be measured by the appended claims and their equivalents.

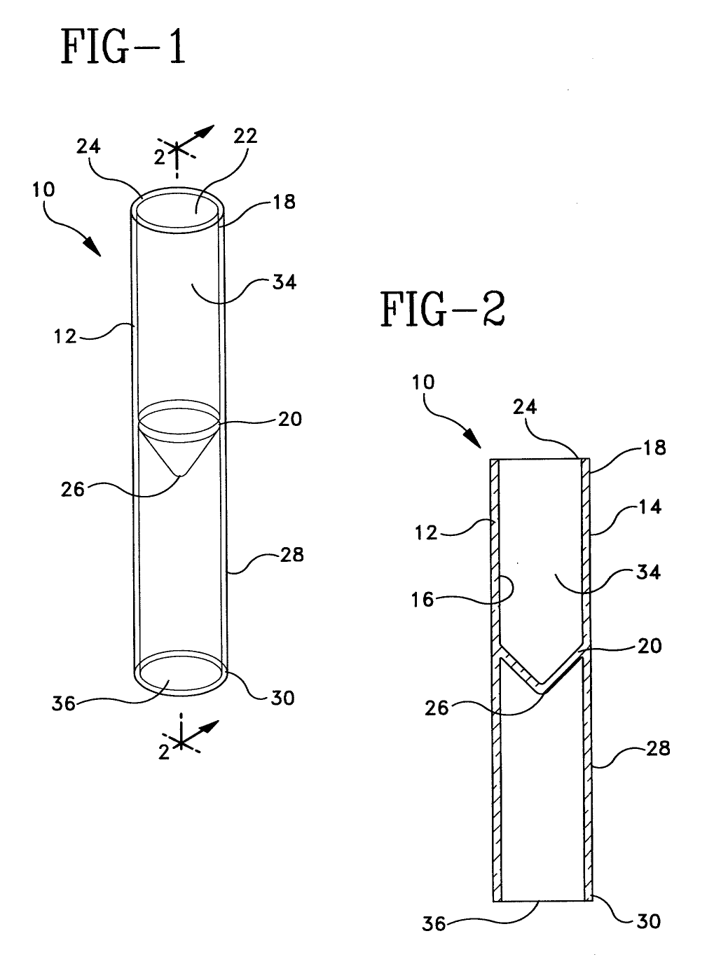

Referring to the drawings in which like reference characters refer to like parts throughout the several views thereof, FIGS. 1 and 2 show a false bottom specimen container 10 of the prior art, having a sidewall 12 having an outer surface 14 and inner surface 16. Sidewall 12 extends from upper portion 18 to lower portion 20. Upper portion 18 includes an open end 22 and a rim 24. Lower portion 20 comprises a closed bottom end 26. An annular skirt 28 extends from lower portion 20 and outer surface 14 to a flat planar bottom end 30 to define a false bottom 32. Interior volume 34 extends between rim 24 and closed bottom end 26.

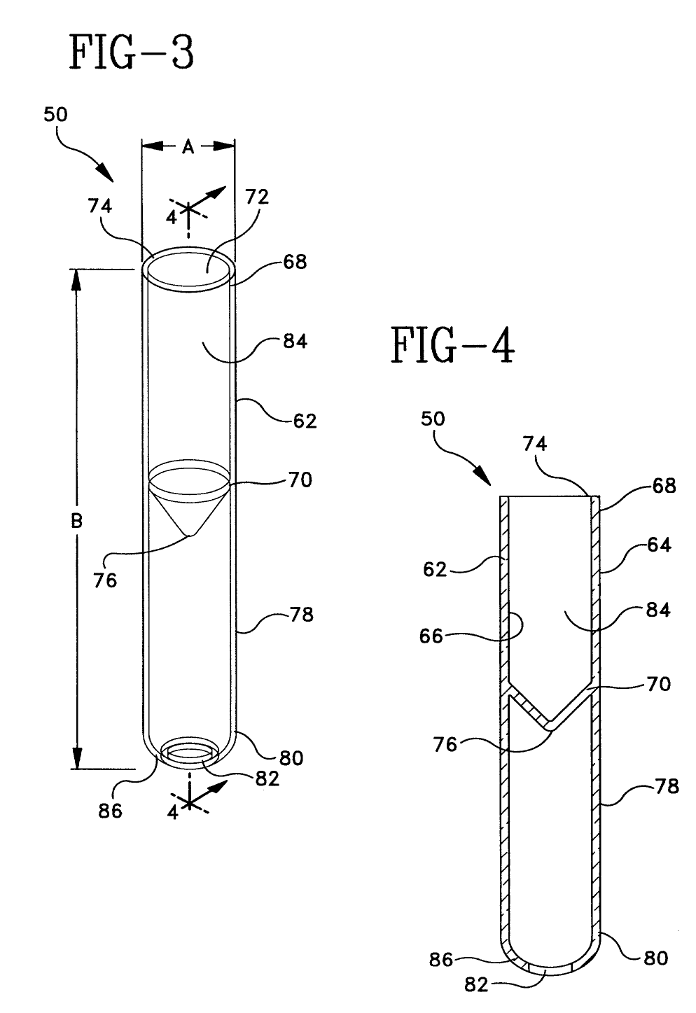

Referring to the drawings in which like reference characters refer to like parts throughout the several views thereof, FIGS. 3 and 4 show the preferred embodiment of the present invention, assembly 50. Assembly 50 is false bottom specimen container, having a sidewall 62 having an outer surface 64 and inner surface 66. Sidewall 62 extends from upper portion 68 to lower portion 70. Upper portion 68 includes an open end 72 and a rim 74. Lower portion 70 comprises a closed bottom end or true bottom 76. An annular skirt 78 extends from lower portion 70 and outer surface 64 to a rounded open bottom end or false bottom end 80 to define an open false bottom area 82. Interior volume 84 extends between rim 74 and closed bottom end 76.

Closed bottom end 76 may be positioned at any point below rim 74 thus providing a variable interior volume 84. Closed bottom end 76 may be generally flat or planar in shape to provide a flat bottom surface for interior volume 84. Alternatively, closed bottom end 76 may be arcuate in shape to provide at least a partially rounded bottom surface for interior volume 84. Most preferably, closed bottom end is generally conical in shape to provide a conical, pointed bottom surface for interior volume 84. Additionally, closed bottom end 76 may be integral with sidewall 62 or may be a discrete member. Preferably closed bottom end 76 is integrally formed with sidewall 62.

Rounded open bottom end 80 includes arcuate shoulder 86. Arcuate shoulder 86 provides the assembly with a curved arcuate, or at least a partially rounded false bottom end 80. The false bottom end provides for better compatibility with clinical equipment and analytical or diagnostic testing equipment or instruments which are designed to accommodate conventional standard-sized round bottom blood collection tubes.

Container 50 has an outer diameter A of about 13 millimeters, a length B of about 75 millimeters, as measured from rim 74 to arcuate shoulder 86 of rounded open bottom end 80, and an interior volume 84 of about 1 to 3 millimeters. It is within the purview of this invention that container 50 may have an outer diameter of about 13 to about 16 millimeters, a length of about 75 to about 100 millimeters and an interior volume of about 1 to about 3 milliliters.

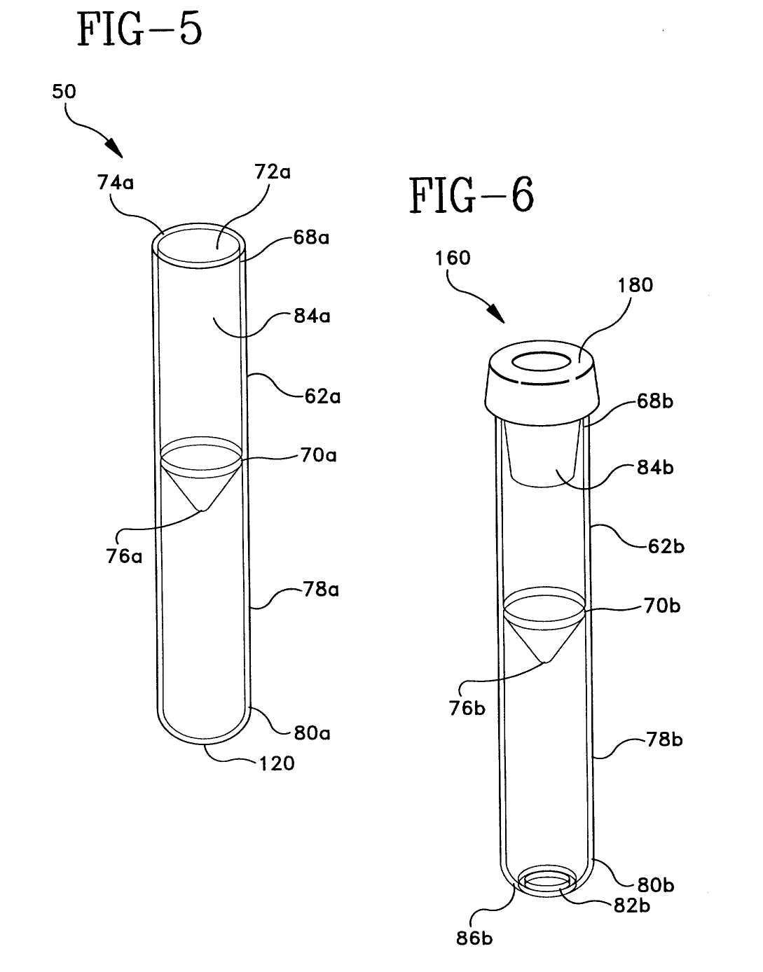

The invention, as shown in FIG. 5 includes many components which are substantially identical to the components of FIGS. 3-4. Accordingly, similar components performing similar functions will be numbered identically to those components of FIGS. 3-4, except that a suffix “a” will be used to identify the similar components in FIG. 5.

As illustrated in FIG. 5, a further embodiment of the invention is assembly 100, wherein annular skirt 78a extends from closed bottom end 76a and outer surface 74a to a rounded closed bottom end or false bottom 120. Rounded closed bottom end 120 is essentially a rounded or semi-spherical shape. Assembly 100 with the rounded closed bottom end or false bottom end is compatible with clinical equipment or instruments which are designed to accommodate conventional standard-sized round bottom blood collection tubes.

The invention, as shown in FIG. 6 includes many components which are substantially identical to the components of FIGS. 3-4. Accordingly, similar components performing similar functions will be numbered identically to those components of FIGS. 3-4, except that a suffix “b” will be used to identify the similar components in FIG. 6.

As illustrated in FIG. 6, a further embodiment of the invention is assembly 160 which includes a cap 180.

The embodiment of FIG. 6 may be evacuated or non-evacuated. When assembly 160 is evacuated, it has a full-draw internal pressure so as to be able to draw a sufficient quantity of blood to substantially fill interior volume 84b. Interior volume 84b is typically maintained at a lower-than-atmospheric internal pressure so that when a blood collection probe penetrates through the cap placing interior volume 84b in communication with the circulatory system of a patient, the lower-than-atmospheric pressure of interior volume 84b will draw blood from the patient into the tube. Assembly 160 may be described as a full-draw blood collection tube because the internal pressure of interior volume 84b is low enough to draw a volume of blood substantially equal to the volume of interior volume 84b.

The various embodiments of the present invention may be manufactured by known manufacturing methods including but not limited to injection molding or according to the following method:

a. providing an elongated tubular housing having opposed first and second ends and a cylindrical wall therebetween which defines a tubular interior;

b. positioning a solid partition within the tubular housing between the first and second ends;

c. heating a forming tool or die that may be constructed of metal or durable conductive material to about 40° C. to about 125° C. and most preferably at about 70° C.;

d. inserting one of the ends of the tubular housing into the forming tool that has an arcuate shaped recess such as spherical;

e. applying a force of about 25 to about 400 pounds per tube for about 3 to about 7 seconds to the tubular housing to cause the end to soften and to conform or assume the arcuate shaped recess of the forming tool; and

f. removing the tubular housing from the forming tool and cooling at about 70° F. for about 10 seconds.

What is claimed is:

1. A collection container assembly comprising:
an elongate tubular housing having opposed first and second ends and a cylindrical wall therebetween defining a tubular interior; and
a solid partition positioned within said housing between said first and second ends;
said housing defining a volume for specimen collection therein between said first end and said partition,
said second end being reconfigured into at least a partially arcuate shape to provide said specimen collection tube with at least a partially rounded end.

2. The assembly of claim 1, wherein said second end is open to an interior portion of said housing.

3. The assembly of claim 1, wherein said second end is closed to define a fully rounded end.

4. The assembly of claim 1, wherein said partition is integral with said housing.

5. The assembly of claim 1, wherein said partition is arcuate in shape to provide said volume for specimen collection with at least a partially rounded bottom portion.

6. The assembly of claim 1, wherein said partition is conical in shape.

7. The assembly of claim 1, wherein said housing is a thermoplastic polymer.

8. The assembly of claim 7, wherein said thermoplastic polymer is polyethylene terephthalate, polypropylene, polyethylene napthalate, polyvinyl chloride or copolymers thereof.

9. The assembly of claim 1, wherein said housing comprises an outer diameter, a length and an internal volume, wherein said outer diameter is about 13 to about 16 millimeters, said length is about 75 to about 100 millimeters and said internal volume is about 1 to about 3 milliliters.

10. A method of providing a collection container assembly with an arcuate shaped end comprising:
providing an elongate tubular housing having opposed first and second ends and a cylindrical wall therebetween defining a tubular interior;
providing a solid partition within said housing between said first and second ends; and
reconfiguring one of said ends into at least a partially arcuate shape.

11. The method of claim 10, wherein said housing is a thermoplastic polymer.

12. The method of claim 11, wherein said thermoplastic polymer is polyethylene terephthalate, polypropylene, polyethylene napthalate, polyvinyl chloride or copolymers thereof.

13. The method of claim 11, wherein said reconfiguring step further includes
inserting one of said ends into a forming tool having an arcuate shaped recess; and applying a force to said housing to cause said one of said ends to conform to the shape of said arcuate shaped recess.

14. The method of claim 13, wherein said forming tool is heated prior to inserting one of said ends therein.

15. The method of claim 14, wherein said forming tool is heated to a temperature of about 40° C. to about 125° C.

16. The method of claim 11, wherein said force is applied at a pressure of about 25 to about 400 pounds per tube.

17. The method of claim 11, wherein said force is applied for a period of about 3 to about 7 seconds.

18. The method of providing a collection container assembly with an arcuate shaped end comprising:
forming an elongate tubular housing having opposed first and second ends and a cylindrical wall therebetween defining a tubular interior;
positioning a solid partition within said housing between said first and second ends;
inserting one of said ends into a heated forming tool having an arcuate shaped recess; and
applying a force to said housing to cause said one of said ends to conform to the shape of said arcuate shaped recess.
